(19) United States
(12) Patent Application Publication
(10) Pub. No.: US 2016/0319269 A1
(43) Pub. Date: Nov. 3, 2016

(54) Light Inducible Promoters and Methods of Using Same

(71) Applicant: Sabic Global Technologies, B.V., Bergen Op Zoom (NL)

(72) Inventors: Xinyao LIU, Thuwal (SA); Duncan ROWE, Thuwal (SA)

(21) Appl. No.: 15/137,566

(22) Filed: Apr. 25, 2016

Related U.S. Application Data
(60) Provisional application No. 62/154,487, filed on Apr. 29, 2015.

(51) Int. Cl. C12N 13/00; C12N 15/74

(52) U.S. Cl. CPC C12N 13/00; C12N 15/74

(57) ABSTRACT

A method comprising (a) introducing a plurality of at least one phototrophic organism to a culture media to create a first mixture; (b) subjecting the first mixture to conditions suitable for growth of the phototrophic organism in the presence of a wavelength converting material to produce a concentrated mixture having a first cell titer; (c) diluting the concentrated mixture to produce a diluted mixture having a second cell titer; and (d) subjecting the diluted mixture to conditions suitable for growth of the phototrophic organism in the absence of the wavelength converting material.

### CROSS-REFERENCE TO RELATED APPLICATIONS

The present application is a non-provisional of and claims priority to U.S. Provisional Patent Application No. 62/154,487 filed Apr. 29, 2015 and entitled “Light Inducible Promoters and Methods of Using Same,” which application is incorporated by reference herein in its entirety.

### TECHNICAL FIELD

The present disclosure generally relates to compositions and methods for the induction of gene expression. More particularly, the present disclosure relates to light-inducible promoters of gene expression.

### BACKGROUND

Bioreactor designs with unique performance characteristics will play a vital role in the economic manufacture of useful biotechnological products from natural and genetically modified cell systems of microbial, mammalian, and plant origin. An important feature of such bioreactors is the ability to balance maintenance of the biological system viability with production of the maximum amount of target product.

For example, when using industrial microorganisms to synthesize products, such as proteins, drugs, or chemicals, a two-step process is usually applied. First, grow the microorganism to a certain fixed biomass without production of the desired product. Second, use an induction factor to trigger production of the target product. The reason for this two-step process is that usually production of a very high-titer (many grams per liter) of a target product will consume a large portion of the energy and carbon resource in the organism's cell, which will very likely impinge upon the growth and viability of the cells. Thus, it is important to be able to control the timing of the onset of production of the target product, ideally at the expensive of the microorganism producing more biomass, but usually when the target product and further cell biomass are produced concomitantly.

Constituent expression of the target product can be detrimental to the viability of the organism, consequently, inducible systems that allow for a temporal and spatial controlled activation of genes and proteins are favored. In some cases, an inducible system can avert “metabolic stagnation” and prolong the overall production period. Many common inducible DNA promoters respond to changes in concentration of transition metals, such as Co²⁺, Cu²⁺, Ni²⁺, Zn²⁺, and Fe²⁺, or to the presence of a metabolite analogue such as the chemicals isopropyl β-D-1-thiogalactopyranoside (IPTG), and anhydrotetracycline (aTc). Metabolite inducers are usually not considered as practical for large-scale cultivation because of their high price. Metal ions as inducers also face problems in there use, as there are challenges to removing them completely from the culture medium. Changing the culture conditions for growth of an organism, such as CO₂ limitation, is mainly suitable for final processes like lysing or harvesting the cells, as limiting carbon will reduce the amount of the target product. Thus, there exists an ongoing need for a level of measurable, tunable, and switchable control of gene promoters.

### SUMMARY

Disclosed herein is a method comprising (a) introducing a plurality of at least one phototrophic organism to a culture media to create a first mixture; (b) subjecting the first mixture to conditions suitable for growth of the phototrophic organism in the presence of a wavelength converting material to produce a concentrated mixture having a first cell titer; (c) diluting the concentrated mixture to produce a diluted mixture having a second cell titer; and (d) subjecting the diluted mixture to conditions suitable for growth of the phototrophic organism in the absence of the wavelength converting material.

Also disclosed herein is a method comprising transforming a plurality of at least one phototrophic organism with a construct comprising a promoter having a sequence selected from the group consisting of SEQ ID No. 1, and SEQ ID No. 2, to generate a transformed phototrophic organism; (b) introducing the transformed phototrophic organism to a media to create a first mixture; (c) subjecting the first mixture to conditions suitable for growth of the transformed phototrophic organism in the presence of a wavelength converting material to produce a concentrated mixture having a first cell titer; (d) diluting the concentrated mixture to produce a diluted mixture having a second cell titer; and (e) subjecting the diluted mixture to conditions suitable for growth of the transformed phototrophic organism in the absence of the wavelength converting material.

Also disclosed herein is a method comprising exposing, for a first period of time, a phototrophic organism to modified natural or artificial sunlight, wherein the modified sunlight is shifted toward the red spectrum by passing the sunlight through a medium comprising a luminescent dye; and exposing, for a second period of time, the phototrophic organism to non-modified natural or artificial sunlight whereby the phototrophic organism is induced to express a desired product.

### BRIEF DESCRIPTION OF THE DRAWINGS

For a more complete understanding of the present disclosure and the advantages thereof, reference is now made to the following brief description, taken in connection with the of like parts.

FIG. 1 is a depiction of the spectral pattern of sunlight passed through polyethylene, and sunlight filtered through a polyethylene matrix with a dispersed perylene-containing dye.

FIG. 2 is a depiction of the relative spectra of sunlight measured for polyethylene films containing two concentrations of a perylene-containing dye.

FIG. 3A and FIG. 3B are plots of the Synechococcus 7002 biomass growth under outdoor conditions.

FIG. 4A and FIG. 4C depict TEM images of Synechococcus 7002 cells grown in polyethylene perylene-containing dye bags vs FIG. 4B and FIG. 4D which depict PE bags without extra CO₂ supply in aeration.

FIG. 5 is a plot of the dry weight analysis against time for Synechococcus 7002 cells grown in polyethylene film bag reactors with and without 0.12% NLR doping.

FIG. 6 is a schematic depiction of the procedure and timeframe for taking Synechococcus 7002 cell samples for RNA isolation.

FIG. 7 is a schematic of a construct for evaluation of a promoter element.

FIG. 8 depicts growth curves for Synechococcus 7002 cells transfected with wild type promoter constructs and mutant promoter constructs.

### DETAILED DESCRIPTION

Disclosed herein are compositions and methods for induced gene expression. In an embodiment, the compositions comprise a promoter of the type disclosed herein, alternatively an inducible promoter of the type disclosed herein. The term “promoter” or “promoter polynucleotide” as used herein refers to a DNA sequence which, when ligated to a nucleotide sequence of interest, is capable of controlling the transcription of the nucleotide sequence of interest into mRNA. A promoter is typically, though not necessarily, located 5′ (e.g., upstream) of a nucleotide of interest (e.g., proximal to the transcriptional start site of a structural gene) whose transcription into mRNA it controls, and provides a site for specific binding by RNA polymerase and other transcription factors for initiation of transcription. “Inducible promoter” refers to promoters that direct gene expression not constitutively, but in a temporal- and/or spatial manner.

The promoters of the present disclosure comprise nucleotides which may be provided, isolated, and/or purified from their natural environment, in substantially pure or homogeneous form, or free or substantially free of other nucleotides and/or nucleic acids of the species of origin. An “isolated” nucleotide or polynucleotide as used herein is also substantially free—at the time of its isolation—of other cellular materials or culture medium when produced by recombinant techniques, or substantially free of chemical precursors when chemically synthesized. Herein “substantially free” refers to the level of other components being present in amounts that do not adversely affect the properties of the disclosed compositions and/or the organisms to which the compositions are introduced. For example, the level of such components may be less than about 10%, alternatively less than about 9, 8, 7, 6, 5, 4, 3, 2, or 1% based on the total amount (by weight or by moles) of such materials in a composition for use in the present disclosure.

A promoter of the type disclosed herein may be generally introduced into a vector (e.g., a cloning vector, or an expression vector, or an expression construct) for convenience of manipulation. In addition, a promoter suitable for use in the present disclosure can include an engineered promoter such as a recombinant or a synthetic promoter. In an embodiment, the promoters of the disclosure are polynucleotides. Where used herein, the term “isolated” encompasses all of these possibilities.

Throughout this application, various publications are referenced. The disclosures of all of these publications and those references cited within those publications in their entireties are hereby incorporated by reference into this application in order to more fully describe the state of the art to which this disclosure pertains. Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described, and incorporated by reference herein, in Sambrook and Russell, 2001 Molecular Cloning, Third Edition, Cold Spring Harbor, Plainview, N.Y.; Sambrook et al., 1989 Molecular Cloning, Second Edition, Cold Spring Harbor Laboratory, Plainview, N.Y.; Maniatis et al., 1982 Molecular Cloning, Cold Spring Harbor Laboratory, Plainview, N.Y.; Wu (Ed.) 1993 Meth. Enzymol. 218, Part I; Wu (Ed.) 1979 Meth Enzymol. 68; Wu et al., (Eds.) 1983 Meth. Enzymol. 100 and 101; Grossman and Moldave (Eds.) 1980 Meth. Enzymol. 65; Miller (Ed.) 1972 Experiments in Molecular Genetics, Cold Spring Harbor Laboratory, Cold Spring Harbor, N.Y.; Old and Primrose, 1981 Principles of Gene Manipulation, University of California Press, Berkeley; Schleif and Wensink, 1982 Practical Methods in Molecular Biology; Glover (Ed.) 1985 DNA Cloning Vol. I and II, IRL Press, Oxford, UK; Hames and Higgins (Eds.) 1985 Nucleic Acid Hybridization, IRL Press, Oxford, UK; and Setlow and Hollaender 1979 Genetic Engineering Principles and Methods, Vols. 1-4, Plenum Press, New York.

The promoters (e.g., polynucleotides) of the present disclosure may be placed in operative association with a second polynucleotide that results in the inducible expression of the second polynucleotide in a suitable organism. As used herein, the terms “in operative association,” “operably linked,” and “associated with” are interchangeable and mean the functional linkage of a promoter and a second polynucleotide on a single nucleic acid fragment in such a way that the production of a second polynucleotide is initiated and mediated by the promoter. In general, polynucleotides that are in operative association are contiguous. Fragments or portions of the disclosed promoter are also encompassed by the present disclosure. By “fragment” or “portion” is meant less than full length of the polynucleotide sequence.

In an embodiment, a promoter of the present disclosure has the sequence set forth in SEQ ID NO:1, alternatively SEQ ID NO:2. Such promoters may be a component of a plasmid and disposed within the plasmid such as to be operably linked to a second polynucleotide in order to control the expression of the second polynucleotide.

In an embodiment, the plasmid is associated with a host organism that is subjected to an external stimulus which results in induction of the promoter. Particularly, the induced promoter may respond to the external stimuli by increasing the organism's expression of the second polynucleotide to which it is operably linked. The level to which the organism's expression of the second polynucleotide is increased can be assessed relative to the level of expression of the second polynucleotide in the absence of a promoter of the type disclosed herein such that the external stimuli results in a fold increase expression of the second polynucleotide ranging from about 2 to about 10, alternatively from about 2 to about 7, or alternatively from about 2 to about 5.

In an embodiment, the promoter is of the type disclosed herein, the organism is a phototrophic organism, and the external stimulus comprises subjecting the phototrophic organism to an altered light spectrum.

The phototrophic organisms can be algae (e.g., green algae, red algae, brown algae, golden algae, etc.), other protists (such as euglena), phytoplankton, bacteria (such as cyanobacteria), or combinations thereof. In an embodiment, the organism is a phototrophic organism such as cyanobacteria. Phototrophic organisms rely on light within the Photosynthetically Active Radiation (PAR) region as a source of energy for photosynthesis. The PAR region is typically referred to as light or radiation having a wavelength between 400 to 700 nanometers. Of this, light having a wavelength of about 400 to 500 nanometers (or blue light) and 600 to 700 nanometers (or red light) are more efficiently used by such plants in the photosynthesis process. By comparison, light having a wavelength between about 500 to 600 nanometers (or green/yellow light) is less efficiently used. Further, light having a wavelength of about 700 to 800 nanometers (or far-red light) promotes petiole elongation while inhibiting germination and rooting in plants. Even further, increasing the red to far-red light (“Red to Far-Red Ratio” or “RTFR”) that a plant receives can be beneficial to the plant's growth and quality.

Cyanobacteria is a phylum of bacteria that obtain their energy through photosynthesis. Cyanobacterial species suitable for use in the present disclosure may be selected from the group consisting of Synechocystis sp. PCC 6803, Anabaena sp. PCC 7120, Thermosynechococcus elongatus BP-1, Gloeobacter violaceus PCC 7421, Microcystis aeruginosa NIES-843, Prochlorococcus marinus SS120, Prochlorococcus marinus MED4, Prochlorococcus marinus MIT9313, Synechococcus sp. WH8102, Synechococcus elongatus PCC 6301, Synechococcus sp. CC9311, Synechococcus sp. PCC 7002, Acaryochloris marina MBIC11017, Prochlorococcus marinus str. NATL2A, Anabaena variabilis ATCC 29413, Synechococcus sp. CC9902, Synechococcus sp. CC9605, Prochlorococcus marinus str. MIT 9312, Synechococcus elongatus PCC 7942, Synechococcus sp. JA-2-3B′a(2-13), Synechococcus sp. JA-3-3Ab, Prochlorococcus marinus str. AS9601, Prochlorococcus marinus str. MIT 9515, Prochlorococcus marinus str. MIT 9303, Prochlorococcus marinus str. NATL1A, Prochlorococcus marinus str. MIT 9301, Synechococcus sp. RCC307, Synechococcus sp. WH 7803, Prochlorococcus marinus str. MIT 9215, Prochlorococcus marinus str. MIT 9211, Cyanothece sp. ATCC 51142, Nostoc punctiforme ATCC 29133, Chlorobium tepidum TLS, Rhodopseudomonas palustris CGA009, Trichodesmium erythraeum IMS101, Cyanothece sp. PCC 7424, Cyanothece sp. PCC 7425, Cyanothece sp. PCC 8801, Arthrospira platensis NIES-39, and combinations thereof. In an embodiment, the cyanobacterial species is Synechococcus sp. PCC 7002.

In an embodiment, a method of the present disclosure comprises transfection of a vector comprising a light inducible promoter of the type disclosed herein operably linked to a second polynucleotide into a cyanobacterial species. In an embodiment, the second polynucleotide encodes for a gene that when expressed results in the cyanobacterial production of a target product. For example, expression of the gene may result in the cyanobacterial production of chemicals such as hydrocarbons. The resulting cyanobacterial species containing the light-inducible promoter operably linked to a second polynucleotide is termed the chassis.

In an embodiment, the method comprises introducing the chassis to a photo-bioreactor. The photo-bioreactor may be any suitable vessel prepared from a wavelength converting material (WCM). WCMs suitable for use in the present disclosure may be designed to provide a light spectral pattern that facilitates the increased growth of the organism (e.g., cyanobacteria) while maintaining some basal level of expression of the second polynucleotide and target product. In an embodiment, the organism is grown in a first vessel (e.g., polymeric container such as a bag) comprising a first WCM having a first light spectral pattern. In an embodiment, the method further comprises exposing the organism to a second light spectral pattern that induces the increased expression of the second polynucleotide and target product. Any suitable methodology may be employed for exposing the organism to a second light spectral pattern. For example, the organism may be transferred from a first vessel comprising a WCM to a second vessel (e.g., polymeric container such as a bag) which either lacks a WCM or comprises a second WCM which differs from the first WCM.

In an embodiment, the method comprises using the WCM to protect the diluted cultures of photosynthetic organisms when starting cultures are inoculated outdoor under strong light conditions. WCMs suitable for use in the present disclosure may be prepared as photo-bioreactors or as covers on photo-bioreactors to enhance the cell viability upon exposure to strong outdoor light at low cell density.

An example of a WCM suitable for use in the present disclosure comprises an organic fluorescent dye or a combination of multiple organic fluorescent dyes and a polymeric matrix, wherein the organic fluorescent dye(s) is/are solubilized in or otherwise physically associated with the polymeric matrix.

Organic fluorescent dye(s) suitable for use in the present disclosure are capable of absorbing light comprising a wavelength of 280 to 650 nm and emitting the absorbed light at a wavelength of 400 to 800 nm. In particular aspects, the WCM and/or organic fluorescent dye(s) is capable of absorbing light comprising a wavelength of 450 to 650 nm and emitting the absorbed light at a wavelength of 550 to 800 nm or are capable of absorbing light comprising a wavelength of 280 to 650 nm and emitting the absorbed light at a wavelength of 400 to 700 nm.

In particular embodiments, the organic fluorescent dye can be a perylene-containing compound. The perylene-containing compound can be a perylene diimide. The perylene diimide can have a structure of:

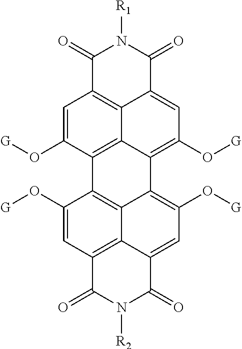

wherein R₁ and R₂ are each independently selected from branched C₆-C₁₈ alkyl and phenyl which is disubstituted by C₁-C₅ alkyl; and G is independently selected from

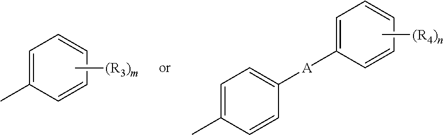

wherein R₃ is independently selected from hydrogen, C₈-C₁₂ alkyl and halogen; m represents the number of substituents and is an integer from 0 to 5; R₄ is independently selected from hydrogen, C₁-C₁₂ alkyl, C₆-C₂₀ aromatic, and C₆-C₂₀ cycloalkyl; n represents the number of substituents and is an integer from 0 to 5; and A is selected from a bond, C₁-C₁₂ alkyl, C₆-C₂₀ aromatic, and C₆-C₂₀ cycloalkyl. Specific non-limiting structures of the perylene dyes are provided in the detailed description and examples section of this specification and are incorporated into this section by reference. In another embodiment, the perylene containing compound can have a structure of

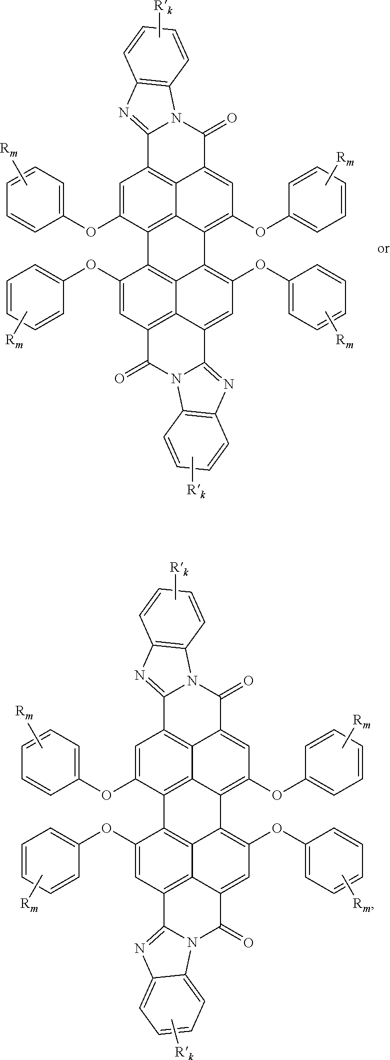

wherein R and R′ are each independently selected from C₈-C₁₈ alkyl, substituted C₈-C₁₈ alkyl, C₈-C₁₈ alkoxy, substituted C₈-C₁₈ alkoxy, and halogen; m represents the number of R substituents on each phenoxy ring, wherein each m is independently an integer from 0 to 5; and k represents the number of R′ substituents on each benzimidazole group, wherein each k is independently an integer from 0 to 4. In certain aspects, the polymeric matrices employed in embodiments (e.g., growth vessels) of the present disclosure can include a combination of various perylene compounds. Also, and in addition to the perylene compounds, the organic fluorescent dye can be a coumarin dye, a carbocyanine dye, a phthalocyanine dye, an oxazine dye, a carbostyryl dye, a porphyrin dye, an acridine dye, an anthraquinone dye, an arylmethane dye, a quinone imine dye, a thiazole dye, a bis-benzoxazolylthiophlnene (BBOT) dye, or a xanthene dye, or any combination of dyes thereof. In certain embodiments, the polymeric matrix can include at least two, three, four, five, six, seven, eight, nine, or ten or more different dyes. In instances where a first and second dye is present in the matrix, the ratio of the first organic fluorescent dye to the second organic fluorescent dye can be from 1:50 to 1:1 to 50:1.

In an embodiment, the polymeric matrix can include a polycarbonate, a polyolefin, a polymethyl (meth)acrylate, a polyester, an elastomer, a polyvinyl alcohol, a polyvinyl butyral, polystyrene, or a polyvinyl acetate, or any combination thereof. In particular embodiments, the polymeric matrix comprises a polycarbonate or a polyolefin or a combination thereof. Examples of polyolefin polymers include polyethylene or polypropylene polymer. Examples of polyethylene polymers include low-density polyethylene polymers, linear low-density polyethylene polymers, or high-density polyethylene polymers. In some aspects, the polymeric matrix can include an additive. Such additives can be used in a variety of ways (e.g., to increase the structural integrity of the matrix or material, to increase the absorption efficiency of the matrix or material, to aid in dispersing the dyes throughout the matrix, to block ultraviolet rays, infrared rays, etc.). In some instances, the additive can be an ultraviolet absorbing compound, an optical brightener, an ultraviolet stabilizing agent, a heat stabilizer, a diffuser, a mold releasing agent, an antioxidant, an antifogging agent, a clarifier, a nucleating agent, a phosphite or a phosphonite or both, a light stabilizer, a singlet oxygen quencher, a processing aid, an antistatic agent, a filler or a reinforcing material, or any combination thereof. An example of an optical brightener is 2,2′-(2,5-thiophenediyl)bis(5-tert-butylbenzoxazole). In certain aspects, the additive can be a diketopyrrolo-pyrrole (DPP) containing compound. Non-limiting examples of DPP compounds include those having the following structure:

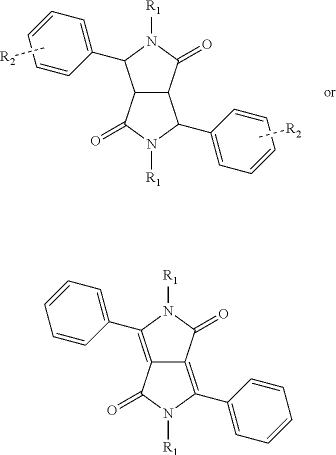

wherein R₁ and R₂ can each individually be H, CH₃, CH₂H₅, 2-ethylhexyl, an amine, or a halogen (e.g., Cl). In particular, embodiments, R₁ and R₂ are each hydrogen. In other instances, R₁ can be hydrogen and R₂ can be a halogen such as Cl. Other derivatives of DPP can also be used in the context of the present invention such that the R₁ and R₂ groups can be C₁ to C₈ linear and branched alkyl groups, phenol groups, etc. In some embodiments, the additive can be a pigment. In other embodiments, the polymeric matrix does not include a pigment or does not include a perylene-based pigment. The polymeric matrix or wavelength converting material (e.g., as employed in a growth vessel or container) can be designed such that it is also capable of absorbing ultraviolet light comprising a wavelength of 280 to 400 nm. In such cases, the polymer matrix can further include an ultraviolet light absorbing compound that is capable of absorbing ultraviolet light comprising a wavelength 280 to 400 nm. In particular instances, the ultraviolet light absorbing compound is capable of emitting said absorbed light in the range of 400 to 800 nm or 400 to 500 nm, or 600 to 700 nm, or 600 to 800 nm. The ultraviolet light absorbing compound can be capable of absorbing ultraviolet light comprising a wavelength of 315 to 400 nm, wherein said compound can be avobenzone (Parsol® 1789, DSM, Switzerland), bisdisulizole disodium (Neo Heliopan® AP, Symrise AG, Germany), diethylamino hydroxybenzoyl hexyl benzoate (Uvinul® A Plus, BASF), ecamsule (Mexoryl™ SX), or methyl anthranilate, or any combination thereof. Avobenzone is also known as methoxydibenzoylmethane and ecamsule is also known as terephthalylidene dicamphor sulfonic acid. The ultraviolet light absorbing compound can be capable of absorbing ultraviolet B light comprising a wavelength of 280 to 315 nm, wherein said compound can be 4-aminobenzoic acid (PABA), cinoxate (2-ethoxyethyl p-methoxycinnamate), ethylhexyl triazone (Uvinul® T 150), homosalate (3,3,5-trimethylcyclohexyl 2-hydroxybenzoate), 4-methylbenzylidene camphor (Parsol® 5000), octyl methoxycinnamate (octinoxate), octyl salicylate (octisalate), padimate 0 (2-ethylhexyl 4-(dimethylamino)benzoate, Escalol® 507, Ashland, Inc.), phenylbenzimidazole sulfonic acid (ensulizole), polysilicone-15 (Parsol® SLX), trolamine salicylate. The ultraviolet light absorbing compound can be capable of absorbing ultraviolet A and B light comprising a wavelength of 280 to 400 nm, wherein said compound can be bemotrizinol (Tinosorb™ S, BASF, USA), benzophenones 1 through 12, dioxybenzone, drometrizole trisiloxane (Mexoryl™ XL), iscotrizinol (Uvasorb® HEB, BASF, USA), octocrylene, oxybenzone (Eusolex® 4360, Merck, KGaA, Germany), or sulisobenzone.

The WCM can be configured such that it is placed between a light source (e.g., sunlight, artificial light source (e.g., UV lamp), or a combination of sunlight and artificial light source) and a plurality of at least one phototrophic organism. The plurality of the at least one phototrophic organism (e.g., cyanobacteria) can be included in a liquid medium such as one that includes water. The WCM can be configured to form at least a portion of a container (e.g., polymeric container such as a tub, bag, tank, silo, barrel, bin, tote, etc.) that is configured to hold the liquid medium comprising the plurality of the at least one phototrophic organism. Alternatively, the WCM can be a thin sheet or film that is placed over, adjacent, or otherwise proximate to the container or surrounds the container to provide the wavelength varying effects disclosed herein. In another embodiment, the phototrophic organism can be supported by a substrate (e.g., solid substrate, semi-solid substrate such as a gel substrate, etc.) and a biofilm can be formed by the phototrophic organism. The biomass or biofilm can be formed by subjecting the phototropic organism to light that has been converted by the WCM. In an embodiment, the WCM forms a filter and/or lens for receiving sunlight, modifying the wavelengths thereof, passing the modified light to a target container (e.g., bioreactor) holding a chassis (e.g., a phototrophic organism).

The WCM and/or polymeric matrix can have a stoke shift of 60 to 120 nanometers. In an embodiment, the WCM can be formed into any suitable article for use in the growth of a phototrophic organism (e.g., container, film, sheet, etc.). A film or sheet can be a single-layered or multi-layered film. The film or sheet can be adhered to another. In particular aspects, it is either transparent or translucent. The film or sheet can have a thickness of 10 to 500 μm or from 0.5 to 3 mm. The polymeric matrix or WCM may be thermally stable at a temperature from 200° C. to 350° C. The polymeric matrix or WCM may be capable of emitting more of the absorbed light at a wavelength of 600 to 700 nm than at a wavelength of 700 to 800 nm, thereby increasing the red to far red ratio of the emitted light. In some instances, the polymeric matrix can further include a diffuser such as cross-linked siloxane particles. Non-limiting examples of which include the Tospearl® series diffusers that are commercially available from Momentive Performance Materials, Inc. (e.g., Tospearl® 120, Tospearl® 130, Tospearl® 240, Tospearl® 3120, or Tospearl® 2000.). The diffuser can be an inorganic material comprising antimony, titanium, barium, or zinc, or oxides thereof, and mixtures thereof. In some instances, the organic fluorescent dye is not present on, attached to, or incorporated in silicone flakes or wherein the matrix is not present on, attached to, or incorporated in silicone flakes.

In an embodiment, the chassis (e.g., cyanobacteria comprising a promoter operably linked to a second polynucleotide) is grown in a WCM that was fabricated into a container or vessel (e.g., a polymeric bag bioreactor). In an embodiment, the chassis (e.g., cyanobacteria comprising a promoter operably linked to a second polynucleotide) is grown in a vessel that was covered by a WCM (e.g., a polymeric sheet or film.) In an embodiment, the WCM comprises a polyethylene matrix having a perylene-containing dye dispersed therein. The chassis may be grown to any suitable biomass and then subjected to an altered spectral pattern that induces expression of the second polynucleotide. For example, the chassis may be transferred to a second vessel and grown in the absence of the perylene-containing dye, or alternatively grown in the presence of natural sunlight. For another example, the chassis may be first grown in a vessel that is covered by a WCM film, and then exposed to the full sunlight spectral pattern by removing the WCM film. In an embodiment, the chassis has been exposed to alterations in the light spectral pattern, for example by being transferred from a vessel associated with a first WCM to a vessel associated with a second WCM or from a vessel associated with a first WCM to a vessel lacking a WCM.

In an embodiment, the chassis is a cyanobacterial species comprising a promoter operably linked to second polynucleotide and is grown to a desired biomass or titer (number of cells per unit volume) in a WCM comprising a perylene-containing dye. The chassis grown in the WCM may express a target product at level a. The chassis having reached the some user and/or process desired biomass may be transferred to a second container and subjected directly to sunlight which induces the promoter to increase expression of the second polynucleotide resulting in production of the target product at level 13 where 13 is greater than a.

Without wishing to be limited by theory, the WCM comprising a perylene-containing dye may exert a “protective effect” at the early growth stages of the cyanobacterial cells which are low in number, and hence exposed to more light. As the cell number increases, an effect called self-shading increases where the light intensity reaching an individual cell is reduced. Utilization of the perylene-containing dye in the WCM reduces the amount of high-energy blue light and thus protects diluted cultures from light damage and as a consequence may reduce the occurrence of ‘culture crash’, whereby cells will very quickly die.

An embodiment of a method of the type disclosed herein comprises transforming a phototrophic organism (e.g., cyanobacteria) with a construct comprising a light-inducible promoter. The light inducible promoter may have the nucleotide sequence set forth in SEQ ID No. 1; alternatively SEQ ID No. 2. Herein transformation refers to the genetic alteration of a cell resulting from the direct uptake and incorporation of exogenous genetic material (exogenous DNA) from its surroundings and taken up through the cell membrane(s). The transformed phototrophic organism may be introduced to an appropriate culture media under conditions suitable for growth of the transformed phototrophic organism in the presence of a WCM of the type disclosed herein. Culture medium is a liquid or gel designed to support the growth of microorganisms or cells. In an embodiment, an inoculum of the transformed phototrophic organism is grown to a high titer culture in the presence of the WCM. The method may further comprise diluting the high titer culture with an appropriate diluent (e.g., culture media) and growing the diluted culture in the absence of the WCM. As disclosed herein, growth in the absence of the WCM results in induction of the light-inducible promoter, elevated expression of the second polynucleotide, and generation of a target product.

In an embodiment, the methods disclosed herein may be utilized to control gene expression in cyanobacteria on an industrial scale. Particularly, cyanobacteria may be grown in association with a WCM comprising a perylene-containing dye which may facilitate the acclimatization of indoor cyanobacteria cultures to outdoor conditions. In an embodiment, the WCM is associated with a cyanobacteria culture during a transaction stage such as during the initial growth of the cyanobacteria. Inoculums (e.g., 10 liters) may first be associated with a WCM comprising a perylene-containing dye (e.g., 100 liters) for acclimating to outdoor conditions. Then the acclimated transaction inoculum (e.g., 100 liters) may be diluted into large volume cultivation in a vessel in absence of a perylene-containing dye (e.g., 1000 liters). In an embodiment, this transaction process (change of sunlight spectrum profile and culture dilution) may be utilized as an inducing factor for induction of production pathway genes.

Further disclosed herein are methods for the growth of cyanobacteria with and without the WCM comprising a perylene-containing dye in order to identify promoters that respond to the change from predominantly red light to white light.

### EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Example 1

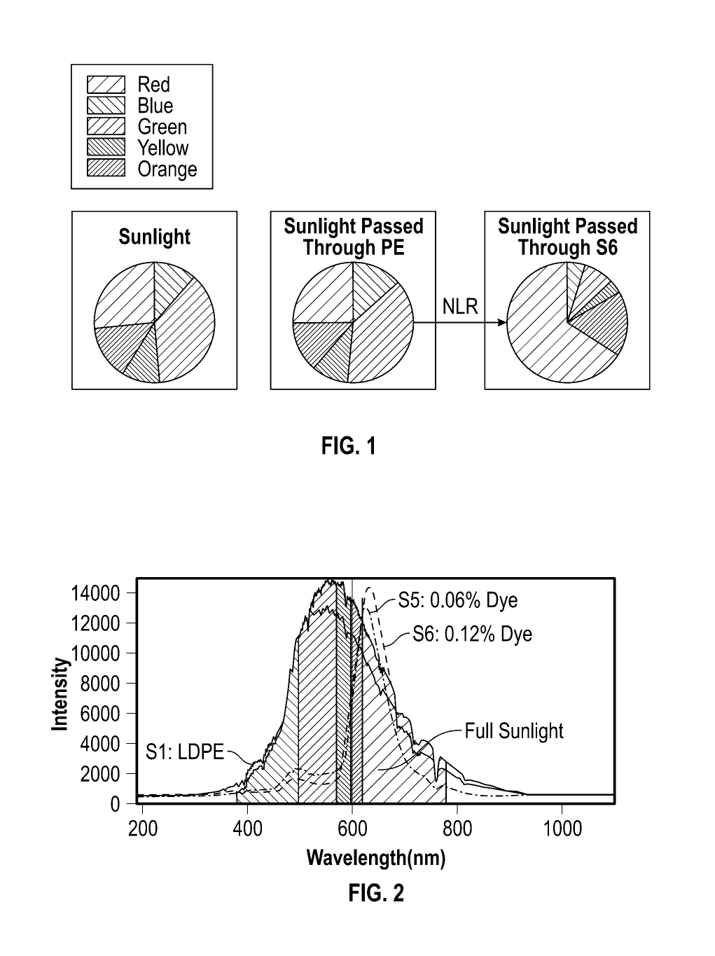

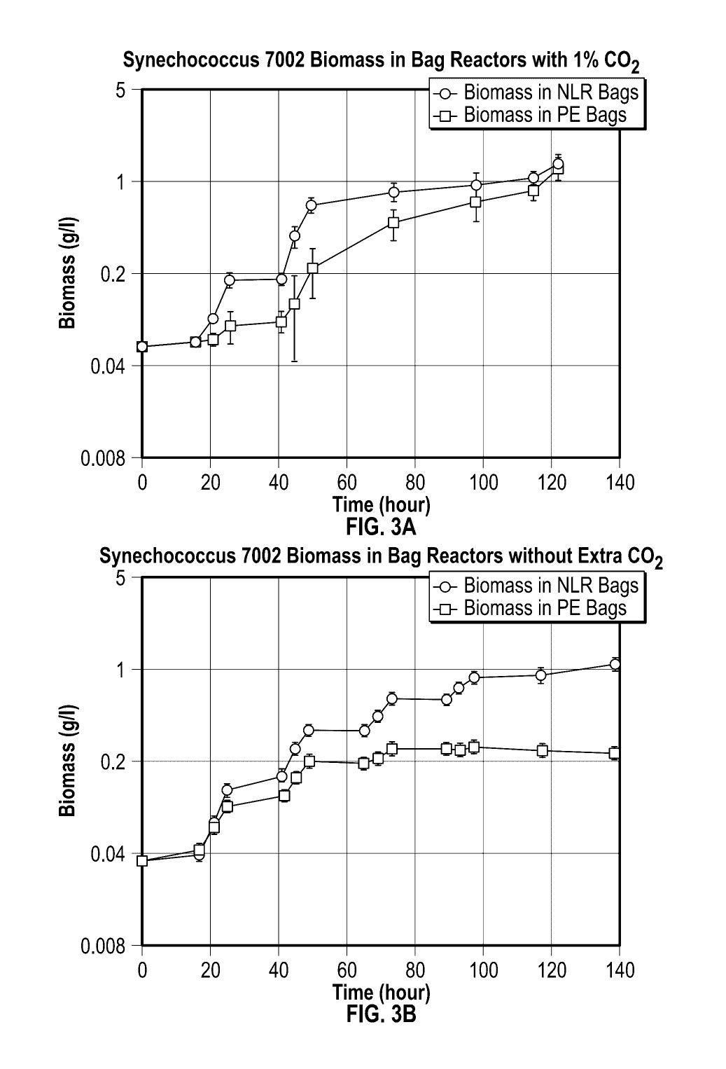

The shift in the spectrum of sunlight caused by perylene-containing dyes dispersed in polyethylene films (PE) were investigated. Specifically, a luminescent perylene-containing dye, designated NLR dye, was synthesized by SABIC Technology Center-Bangalore, India, and incorporated into PE which was the extruded into 0.1-mm thick polyethylene (PE, hereafter) roll (38 cm in width). The shaping force of PE films extruded with NLR dye on sunlight was determined and the results are given in Table 1 and FIGS. 1 and 2 (designated S6). Sunlight and sunlight passed through the NLR films were analyzed outdoors at noon, on a sunny day.

Referring to FIG. 2, full sunlight spectrum is indicated by the colorful bell; green bell, sunlight altered by PE film; blue bell, sunlight altered by S5 (PE with 0.06% NLR); red bell, sunlight altered by S6 (PE with 0.12% NLR). NLR films shifted the sunlight spectrum towards red. The full light intensity (400-700 nm) was measured by light meter LI-250A (LI-COR).

The solar spectra (natural or shifted by the red films) were measured by a spectrometer HR2000+ES (OceanOptics), and the solar intensities were measured by light meter LI-250A (LI-COR). The data suggested the NLR films reduced blue light (e.g., 67% of blue in sunlight by S6); the NLR films increased red light (e.g., 100% of red in sunlight by S6) and the NLR films also increased the lights absorbed photosynthetic pigments (Chl a, Chl b and phycocyanin).

TABLE 1

| | Intensity change % of sunlight chromatography | | |
|---|---|---|---|
| | | S5 | S6 |
| | S1 | PE + | PE + |
| | Normal PE | 0.06% NLR | 0.12% NLR |
| Blue 450-495 nm | 1.9 | −55 | −67 |
| Green 495-570 nm | −10 | −72 | −82 |
| Yellow 570-590 nm | −17 | −58 | −71 |
| Orange 590-620 nm | −18 | 21.4 | 0.1 |
| Red 620-700 nm | −18 | 73.7 | 100 |
| Chl a 650-700 nm | −18 | 27.7 | 51.1 |
| (algae/cyanobacteria) | | | |
| Chl b 600-650 nm | −18 | 75 | 77.6 |
| (algae) | | | |
| Phycocyanin | −18 | 72.7 | 92.8 |
| 610-700 nm | | | |
| (cyanobacteria) | | | |

### Example 2

The protective effects of PE and PE-NLR bags on Synechococcus sp. PCC 7002 cultures under outdoor conditions were investigated. Bags were fabricated with PE or PE-NLR films to grow Synechococcus sp. PCC 7002 under outdoor conditions. The bags were made by a sealing machine with working volume between 700-1,200 ml. The bottoms of bags were sat in a water reservoir for cooling that was sufficient to keep the culture temperature below 36-39° C.

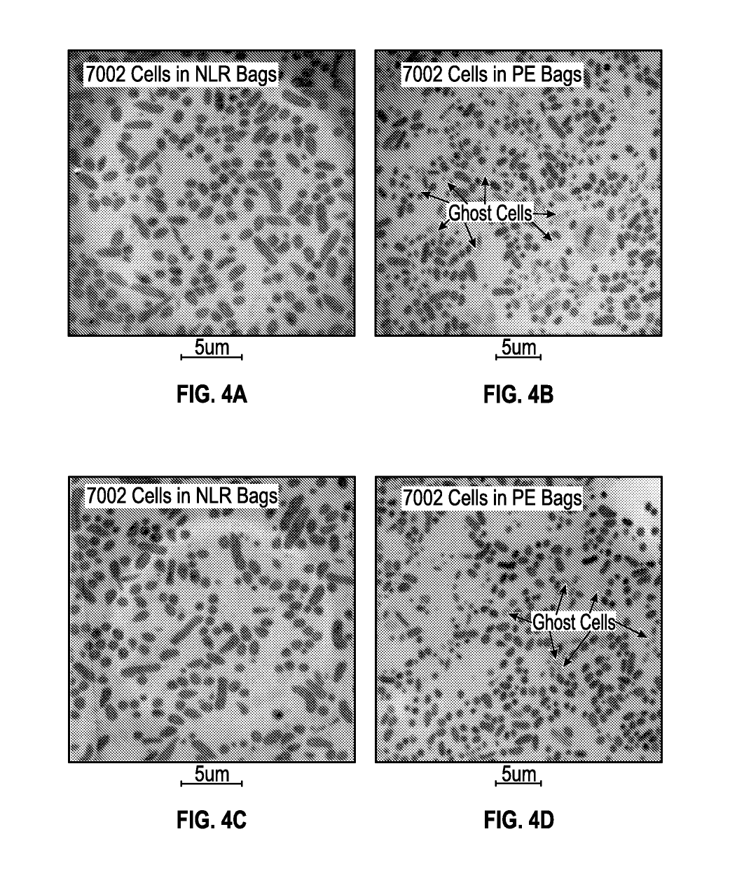

The PE-NLR film (S6) reduced the lag phase and promoted the initial growth of Synechococcus sp. PCC 7002 cultures when the indoor inoculums were first inoculated under the outdoor conditions (FIGS. 3A and 3B). In the first 100 hours, the Synechococcus biomass with 1% CO₂ supply in the NLR bags were higher than those in the PE bags (FIG. 3A). The difference was even bigger when using aerated air without extra CO₂ supply (FIG. 3B). In the first 50 hours, photosynthetic efficiency of the NLR bags (2.26%) is 3-fold higher than that of PE bags (0.74%), suggesting the PE-NLR films significantly reduced the photo-inhibition during the initial acclimation stage. TEM images, FIGS. 4A and 4C, showed that Synechococcus cells grown in NLR bags had a better cell integrity than the cells grown in blank PE bags, FIGS. 4B and 4D. In FIGS. 4B and 4D (cells grown in PE bags), many “ghost” cells (lysed dead cells with only a cell wall structure) are found as an indication of low cell viability for low-density cultures upon inoculation under outdoor conditions. These observations suggested that the red films (i.e. PE-NLR bags) helped the indoor Synechococcus cultures acclimate to the outdoor conditions.

### Example 3

The identity of the light inducible genes responding to a change in the profile of sunlight was investigated. Specifically in order to understand the transcriptome profile and the changes of Synechococcus PCC7002 in NLR and PE bags, RNAseq analysis on the cells grown in Example 2 was performed. The cyanobacterium Synechococcus sp. PC

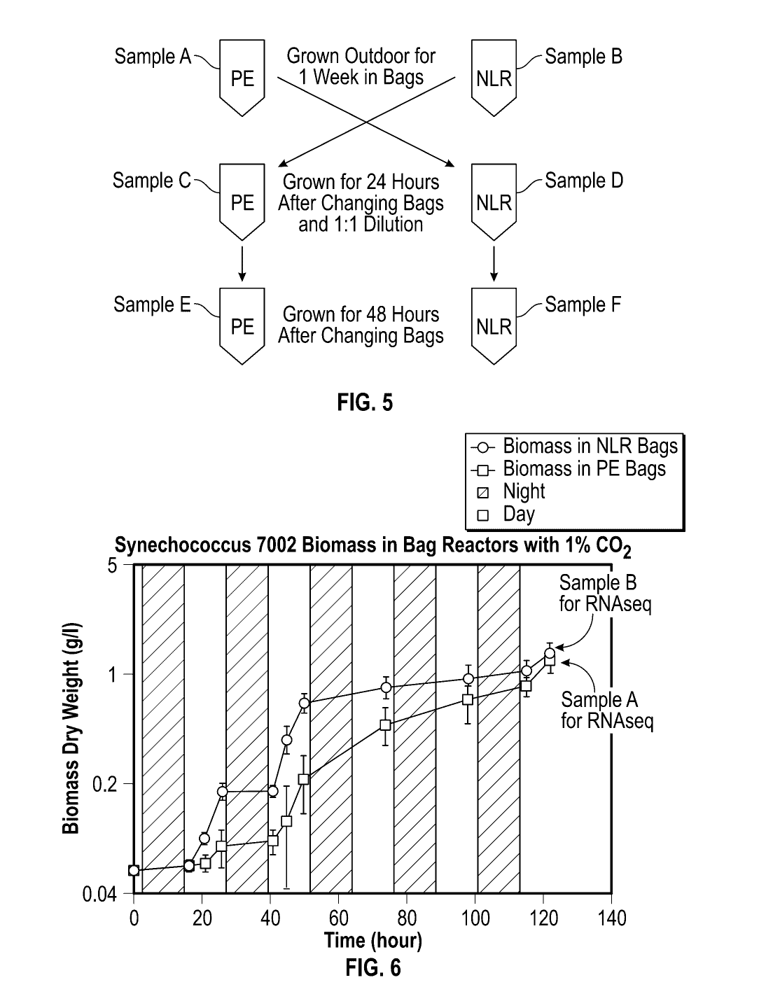

C7002 was cultured in Sea-water MN supplemented with 0.6 g/l urea and grown outdoors where sunlight intensity ranged from 0-1800 μmol photons m⁻² s⁻¹. Cultures were grown in six 1.3-liter fabricated PE for PE-NLR bags in triplicate using air supplemented with 1% (v/v) CO₂. Temperature control was maintained by using a water sink which kept the culture temperature between 36-39° C. The cultures in PE-NLR bags showed better growth than those in the PE bags in the first 120 hours, FIG. 5.

Sample A was directly taken from the PE bags (grown one week outdoor, ˜1 g/l of dry weight), and Sample B was taken from the PE-NLR bags (grown one week outdoor, ˜1 g/l of dry weight). Then the cultures in PE-NLR bags and PE bags were taken out, diluted at 1:1 by fresh sea-water MN media, and switched to the opposite bags. One day after switching the bags, Sample C was taken from the PE bags, and Sample D was taken from the PE-NLR bags. Two days after switching the bags, Sample E was taken from the PE bags, and Sample F was taken from the PE-NLR bags. This process is schematized in FIG. 6.

Each sample was extracted using RiboPure-Bacteria kit (Life Technologies). cDNA library construction, Illumina sequencing, and data analysis were following the protocol of Ludwig [Marcus Ludwig and Donald A. Bryant (2011) Frontiers in Microbiology, Vol 2, Article 41]. T-test was used to compare the normalized RNA transcription data of samples B vs. A, C vs. B, E vs. B, D vs. A, and F vs. A.

Each sample was divided into three for RNA extraction by RiboPure-Bacteria kit (Life Technologies). A comparison was made to the normalized RNA transcription data of samples B vs. A, C vs. B, E vs. B, D vs. A, and F vs. A. Then, based on three criteria: 1) low expression in PE-NLR and high in PE bags, 2) increase expression when transferred from PE-NLR bags to PE bags, and 3) decrease in expression when transferred from PE bags to PE-NLR bags. A top ten out of 3234 genes from the Synechococcus 7002 genome were identified as ‘high responders’, (Table 2, ten genes A2137 D0016, D0011, D0017, petM, A2740, ispA, tig, A3012, and psaE). T-test was used to compare the normalized RNA transcription data of samples B vs. A, C vs. B, E vs. B, D vs. A, and F vs. A. Genes cpcA and A1591 are examples of increase with dilution, gene rbcL is a control for stable promoters.

TABLE 2

| | NLR over | | NLR to | | NLR to PE, | | PE to NLR, | | PE to NLR, | | | |
|---|---|---|---|---|---|---|---|---|---|---|---|---|
| | PE | | PE, 1 day | | 2 days | | 1 day | | 2 days | | | |
| | (B vs A) | | (C vs B) | | (E vs B) | | (D vs A) | | (F vs A) | | gene | SGFP |
| gene ID | Diff. | fold | Diff. | fold | Diff. | fold | Diff. | fold | Diff. | fold | annotation | mutant |
| A2137 | −1542 | −3.63 | 2367 | 5.04 | 107 | 1.18 | −887 | −1.72 | −1868 | −8.15 | tRNA-Ser | FAB401 |
| D0016 | −139 | −3.39 | 98 | 2.09 | 63 | 2.09 | −68 | −1.53 | −113 | −2.35 | hypothetical | FAB402 |
| D0011 | −570 | −2.49 | 30 | 1.70 | 181 | 1.48 | −465 | −1.95 | −381 | −1.67 | hypothetical | FAB403 |
| D0017 | −579 | −2.44 | 768 | 2.91 | 250 | 1.62 | −70 | −1.08 | −464 | −1.90 | hypothetical | FAB404 |
| petM | −1497 | −2.34 | 1030 | 1.92 | 549 | 1.49 | −98 | −1.04 | −322 | −1.14 | cytochrome | FAB405 |
| | | | | | | | | | | | b6-f complex | |
| A2740 | −710 | −2.33 | 1191 | 3.23 | 565 | 2.06 | 247 | 1.20 | −923 | −3.89 | tRNA-Glu | FAB406 |
| lspA | −1338 | −2.02 | 1681 | 2.29 | 386 | 1.30 | −903 | −1.52 | −1376 | −2.09 | signal | FAB407 |
| | | | | | | | | | | | peptidase II | |
| tig | −767 | −1.97 | 1057 | 2.33 | 376 | 1.47 | −94 | −1.06 | 44 | 1.03 | trigger factor | FAB408 |
| A0312 | −5227 | −1.76 | 7190 | 2.04 | 5326 | 1.77 | −931 | −1.08 | 4392 | 1.36 | hypothetical | FAB409 |
| psaE | −2431 | −1.75 | 3808 | 2.18 | 2119 | 1.66 | 1143 | 1.30 | −796 | −1.16 | photosystem I | FAB410 |
| Control | | | | | | | | | | | | Note |
| cpcA | 17437 | −1.88 | 3907 | 1.1 | −8695 | −1.3 | 32395 | 2.64 | 12159 | 1.6 | phycocyanin | dilution |
| | | | | | | | | | | | | increase |
| rbcL | 703 | −1.96 | −341 | −1.33 | −496 | −1.5 | 411 | 1.6 | 81 | 1.1 | RuBisCO | Stable |
| A1591 | 6500 | 4.92 | 16853 | 3.07 | 11913 | 2.46 | 19072 | 13.49 | 13674 | 9.24 | hypothetical | PE to NLR |
| | | | | | | | | | | | | increase |

### Example 4

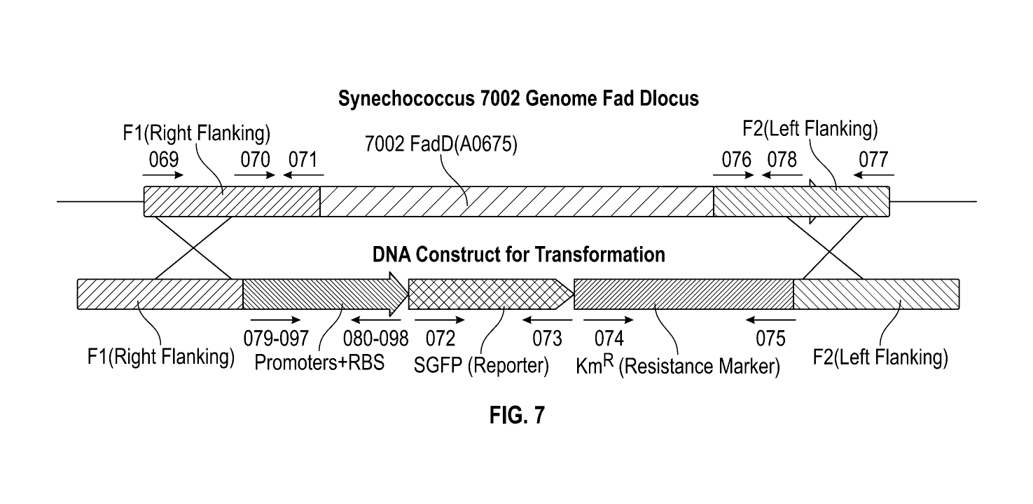

The efficiency of the identified light-inducible promoters was investigated. In order to test the ten promoter containing regions of the light responding genes that were identified by RNAseq analysis as described in Example 3, between 200-500 nucleotides upstream of the start codon of the gene of interest was cloned using the responsive DNA promoter regions in front of the gene for a Super-folded Green Fluorescent Protein (SGFP) as strategy outlined in FIG. 7. It was contemplated that within the 200-500 base paired DNA there will be the promoter element to drive expression of a Super-folded Green Fluorescent Protein (SGFP) that responds to the change in sunlight profile. The SGFP protein was used as an output signal of promoter activity on light stimulation by measuring fluorescence. The DNA reporter cassette was designed to contain the promoter element under investigation cloned in front of the SGFP with a communal ribosome binding site (RBS) linked to a separate kanamycin selectable maker gene and the whole DNA cassette contained flanking regions of homologous DNA to the fadD gene locus found on the chromosome to allow for insertion into the chromosome of Synechococcus 7002, (FIG. 7). The DNA/SGFP reporting cassette was integrated into the Synechococcus 7002 genome by homologous DNA recombination at a selected genome locus called fadD. Referring to FIG. 7, the arrows indicate the primers for construction of the DNA/SGFP reporter cassette by the Polymerase Chain Reaction (PCR). DNA sequences of the primers, genes and light responsive promoter regions for carrying out PCR reactions are referred to in SEQ ID Nos 3-15. The variable fragment is the light responsive “promoter+RBS”, which will have 10 different DNA sequences, but the same RBS.

Each of the 10 PCR DNA fragments were transformed into naturally competent Synechococcus PCC7002 cells. Briefly, 50 μl cells (OD730 nm-1) were mixed with 500 ng of purified PCR DNA for 6 hours at room temperature. The transformed cells were grown in 2 ml Sea-water MN media for 3 days with shaking (180 r.p.m), 100 μmol photons m-2 s-1 at 30° C., and then plated onto Sea-water MN agar plates supplemented with 100 mg/l kanamycin and incubated under the same conditions, but without shaking. The colonies were screened by PCR and the DNA sequenced for confirmation that the DNA/SGFP reporter cassette had been correctly assembled and was present in the cells.

Ten strains were constructed in this manner and were designated: FAB401 for A2137 promoter, FAB402 for D0016 promoter, FAB403 for D0011 promoter, FAB404 for D0017 promoter, FAB405 for petM promoter, FAB406 for A2740 promoter, FAB407 for ispA promoter, FAB408 for tig promoter, FAB409 for A3012 promoter, and FAB410 for psaE promoter.

### Example 5

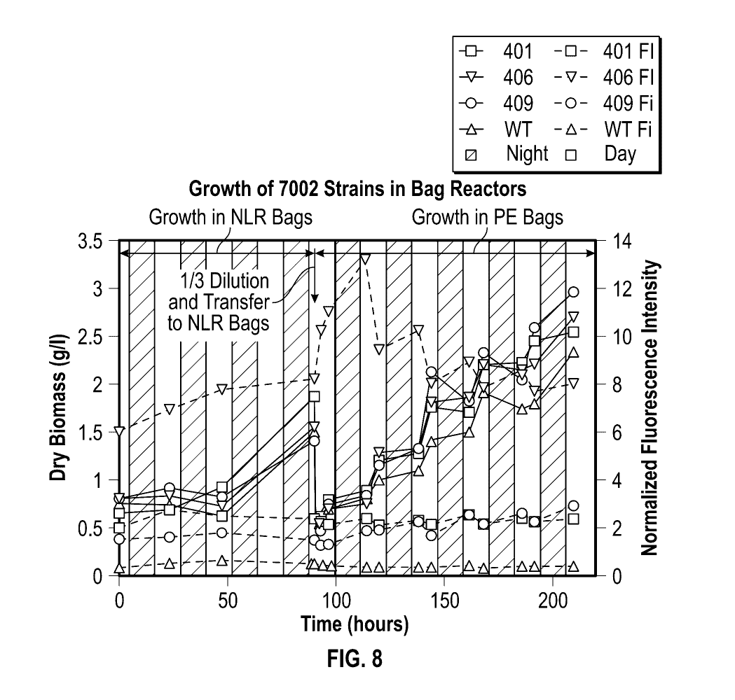

The SGFP fluorescence of the ten strains were measured following dilution and transfer from NLR doped PE film PBRs to PE film PBRS. To test each of the ten light responsive promoters, the level of SGFP fluorescence was measured when each of the FAB401-410 Synechococcus 7002 cells were cultured in 350 ml MN Red Sea salts media with 0.6 g/L urea outdoors with a maximum sunlight intensity of 1,400 μmol photons m-2 s-1, aerated with 1% CO₂, using 0.12% NLR doped PE film bags from 50 ml of inoculation. After 80 hours the acclimatized cultures were diluted to 700 ml with fresh seawater MN media and transferred into PE film PBR bags, and allowed to grow for a further 120 hours. The biomass growth of ten mutant strains and one wildtype strain was similar. Three mutants and the wild type were shown as representatives (filled lines in FIG. 8). Based on the fluorescence intensities of reporter SGFP (dash lines in FIG. 8), the signal levels of all mutants are higher than that of the wild type strain (background), and three types of protein synthesis responses were observed after transfer and dilution. First type, mutant 406 showed a quick increase (1.7-fold) in the first 24 hours, and then the SGFP level started to drop. Second type, mutant 409 showed a slow but steady increase (2.3-fold) in the 120 hours after transfer. Third, all the other mutants did not show significant change in SGFP levels (mutant 401 as an example shown in FIG. 8. The normalized fluorescence intensities of the culture were higher in the morning than in the afternoon. This may be due to “dilution” of SGFP by vigorous cell division during the day. These findings suggest different options for controlling protein synthesis in Synechococcus 7002, such as: quick increase, slow increase, and stable at different levels.

Samples were taken during the experimental period, and analyzed for fluorescence intensity. A PHEARstar FS microplate reader (BMG LABTECH) was used to measure the SGFP fluorescence intensity of the FAB401-410 Synechococcus 7002 cells. Briefly, 200 μl diluted cell cultures with an optical density (OD730 nm) of between 0.2-0.4 were transferred into a black 96-well plate. The excitation wavelength was set to 488 nm, and the emission wavelength was set to 509 nm. To keep the readings in the measure range of the PHEARstar FS microplate reader, the parameter “measurement gain” was set at 1204, and 10% of the actual value was acquired as data. The final fluorescence intensity was normalized against the optical density (OD730 nm) of the diluted FAB401-410 cells at the moment of sampling.

While embodiments of the present disclosure have been shown and described, modifications thereof can be made by one skilled in the art without departing from the spirit and teachings of the disclosure. The embodiments described herein are exemplary only, and are not intended to be limiting. Many variations and modifications of the disclosure are possible and are within the scope of the invention. Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the preferred embodiments of the present invention. The discussion of a reference in the Background is not an admission that it is prior art to the present invention, especially any reference that may have a publication date after the priority date of this application. The disclosures of all patents, patent applications, and publications cited herein are hereby incorporated by reference, to the extent that they provide exemplary, procedural or other details supplementary to those set forth herein.

For the purpose of any U.S. national stage filing from this application, all publications and patents mentioned in this disclosure are incorporated herein by reference in their entireties, for the purpose of describing and disclosing the constructs and methodologies described in those publications, which might be used in connection with the methods of this disclosure. Any publications and patents discussed above and throughout the text are provided solely for their disclosure prior to the filing date of the present application. Nothing herein is to be construed as an admission that the inventors are not entitled to antedate such disclosure by virtue of prior invention.

Unless indicated otherwise, when a range of any type is disclosed or claimed it is intended to disclose or claim individually each possible number that such a range could reasonably encompass, including any subranges encompassed therein. When describing a range of measurements every possible number that such a range could reasonably encompass can, for example, refer to values within the range with one significant digit more than is present in the end points of a range. Moreover, when a range of values is disclosed or claimed, which Applicants intent to reflect individually each possible number that such a range could reasonably encompass, Applicants also intend for the disclosure of a range to reflect, and be interchangeable with, disclosing any and all subranges and combinations of subranges encompassed therein. Accordingly, Applicants reserve the right to proviso out or exclude any individual members of any such group, including any subranges or combinations of subranges within the group, if for any reason Applicants choose to claim less than the full measure of the disclosure.

### ADDITIONAL DISCLOSURE

A first embodiment, which is a method comprising: (a) introducing a plurality of at least one phototrophic organism to a culture media to create a first mixture; (b) subjecting the first mixture to conditions suitable for growth of the phototrophic organism in the presence of a wavelength converting material to produce a concentrated mixture having a first cell titer; (c) diluting the concentrated mixture to produce a diluted mixture having a second cell titer; and (d) subjecting the diluted mixture to conditions suitable for growth of the phototrophic organism in the absence of the wavelength converting material.

A second embodiment, which is the method of the first embodiment wherein the phototrophic organisms comprise algae, euglena, phytoplankton, bacteria, or combinations thereof.

A third embodiment, which is the method of the first embodiment wherein the phototrophic organism is selected from the group consisting of Synechocystis sp. PCC 6803, Anabaena sp. PCC 7120, Thermosynechococcus elongatus BP-1, Gloeobacter violaceus PCC 7421, Microcystis aeruginosa NIES-843, Prochlorococcus marinus SS120, Prochlorococcus marinus MED4, Prochlorococcus marinus MIT9313, Synechococcus sp. WH8102, Synechococcus elongatus PCC 6301, Synechococcus sp. CC9311, Synechococcus sp. PCC 7002, Acaryochloris marina MBIC11017, Prochlorococcus marinus str. NATL2A, Anabaena variabilis ATCC 29413, Synechococcus sp. CC9902, Synechococcus sp. CC9605, Prochlorococcus marinus str. MIT 9312, Synechococcus elongatus PCC 7942, Synechococcus sp. JA-2-3B′a(2-13), Synechococcus sp. JA-3-3Ab, Prochlorococcus marinus str. AS9601, Prochlorococcus marinus str. MIT 9515, Prochlorococcus marinus str. MIT 9303, Prochlorococcus marinus str. NATL1A, Prochlorococcus marinus str. MIT 9301, Synechococcus sp. RCC307, Synechococcus sp. WH 7803 Prochlorococcus marinus str. MIT 9215, Prochlorococcus marinus str. MIT 9211, Cyanothece sp. ATCC 51142, Nostoc punctiforme ATCC 29133, Chlorobium tepidum TLS, Rhodopseudomonas palustris CGA009, Trichodesmium erythraeum IMS101, Cyanothece sp. PCC 7424, Cyanothece sp. PCC 7425, Cyanothece sp. PCC 8801, Arthrospira platensis NIES-39, and combinations thereof.

A fourth embodiment, which is the method of any one of the first through third embodiments wherein the wavelength conversion material comprises an organic fluorescent dye and a polymeric matrix, wherein the organic fluorescent dye is solubilized in the polymeric matrix, and wherein the wavelength-conversion material is capable of absorbing light comprising a wavelength of 280 to 650 nm and emitting the absorbed light at a wavelength of 400 to 800 nm.

A fifth embodiment, which is the method of the fourth embodiment wherein the organic fluorescent dye is a perylene-containing compound.

A sixth embodiment, which is the method of the fourth embodiment wherein the organic fluorescent dye is a coumarin dye, a carbocyanine dye, a phthalocyanine dye, an oxazine dye, a carbostyryl dye, a porphyrin dye, an acridine dye, an anthraquinone dye, an arylmethane dye, a quinone imine dye, a thiazole dye, a bis-benzoxazolylthiophene (BBOT)dye, or a xanthene dye, or any combination of dyes thereof.

A seventh embodiment, which is the method of any one of the fourth through sixth embodiments wherein the polymeric matrix comprises a polycarbonate, a polyolefin such as polyethylene, a polymethyl (meth)acrylate, a polyester, an elastomer, a polyvinyl alcohol, a polyvinyl butyral, polystyrene, or a polyvinyl acetate, or any combination or copolymer thereof.

An eighth embodiment, which is a method comprising:

(a) transforming a plurality of at least one phototrophic organism with a construct comprising a promoter having a sequence selected from the group consisting of SEQ ID No. 1, and SEQ ID No. 2, to generate a transformed phototrophic organism;
(b) introducing the transformed phototrophic organism to a media to create a first mixture;
(c) subjecting the first mixture to conditions suitable for growth of the transformed phototrophic organism in the presence of a wavelength converting material to produce a concentrated mixture having a first cell titer;
(d) diluting the concentrated mixture to produce a diluted mixture having a second cell titer; and
(e) subjecting the diluted mixture to conditions suitable for growth of the transformed phototrophic organism in the absence of the wavelength converting material.

A ninth embodiment, which is the method of the eighth embodiment wherein the promoter is operably linked to a second polynucleotide.

A tenth embodiment, which is the method of the eighth embodiment wherein the promoter is induced in the absence of the wavelength conversion material.

An eleventh embodiment, which is the method of the tenth embodiment wherein induction of the promoter results in an increased expression of the second polynucleotide.

A twelfth embodiment, which is the method of the eleventh embodiment wherein expression of the second polynucleotide is increased by from about 2-fold to about 7-fold.

A thirteenth embodiment, which is the method of the eleventh embodiment wherein the increased expression of the second polynucleotide results in the expression of a target product.

A fourteenth embodiment, which is the method of the thirteenth embodiment wherein the target product comprises a hydrocarbon.

A fifteenth embodiment, which is the method of any one of the eighth through fourteenth embodiments wherein the phototrophic organisms comprise algae, euglena, phytoplankton, bacteria, or combinations thereof.

A sixteenth embodiment, which is the method of any one of the eighth through fifteenth embodiments wherein the wavelength conversion material comprises an organic fluorescent dye and a polymeric matrix, wherein the organic fluorescent dye is solubilized in the polymeric matrix, and wherein the wavelength-conversion material is capable of absorbing light comprising a wavelength of 280 to 650 nm and emitting the absorbed light at a wavelength of 400 to 800 nm.

A seventeenth embodiment, which is the method of the sixteenth embodiment wherein the organic fluorescent dye is a perylene-containing compound.

An eighteenth embodiment, which is the method of the sixteenth embodiment wherein the polymeric matrix comprises a polycarbonate, a polyolefin such as polyethylene, a polymethyl (meth)acrylate, a polyester, an elastomer, a polyvinyl alcohol, a polyvinyl butyral, polystyrene, or a polyvinyl acetate, or any combination or copolymer thereof.

A nineteenth embodiment, which is an isolated DNA construct comprising a promoter having a sequence selected from the group consisting of SEQ ID No. 1, and SEQ ID No. 2, operably linked to a second polynucleotide.

A twentieth embodiment, which is the isolated DNA construct of the nineteenth embodiment further comprising a selectable marker.

A twenty-first embodiment, which is the method of the first embodiment wherein the phototrophic organism in the first mixture shows high cell viability under full sunlight conditions.

A twenty-second embodiment, which is a method comprising: exposing, for a first period of time, a phototrophic organism to modified natural or artificial sunlight, wherein the modified sunlight is shifted toward the red spectrum by passing the sunlight through a medium comprising a luminescent dye; and exposing, for a second period of time, the phototrophic organism to non-modified natural or artificial sunlight whereby the phototrophic organism is induced to express a desired product.

A twenty-third embodiment, which is the method of the twenty-second embodiment wherein the exposing for the first period of time is in a first bioreactor, wherein at least a portion of the first bioreactor is formed from a polymer comprising the dye; and wherein the exposing for the second period of time is in a second bioreactor, wherein the first bioreactor is formed from a polymer substantially free of the dye.

A twenty-fourth embodiment, which is the method of any one of the twenty-second through twenty-third embodiments wherein the first period of time is an incubation period associated with transfer of the phototrophic organism to a location receiving direct sunlight.

A twenty-fifth embodiment, which is the method of the twenty-fourth embodiment wherein the incubation period is further associated with a diluted culture of the phototrophic organism such that the phototrophic organism is incapable of self-shading and susceptible to culture crash.

A twenty-sixth embodiment, which is the method of the twenty-fifth embodiment wherein the start of the second period of time is associated with a achieving a non-diluted culture of the phototrophic organism such that the phototrophic organism is capable of self-shading and less susceptible to culture crash than during the first period of time.

A twenty-seventh embodiment, which is the method of any one of the twenty-second through twenty-sixth embodiments wherein the phototrophic organism photosynthetic efficiency is at least 1×, 2×, or 3× greater in the first 50 hours after inoculation than in an otherwise similar first bioreactor absent the dye.

What is claimed is:

1. A method comprising:
(a) introducing a plurality of at least one phototrophic organism to a culture media to create a first mixture;
(b) subjecting the first mixture to conditions suitable for growth of the phototrophic organism in the presence of a wavelength converting material to produce a concentrated mixture having a first cell titer;
(c) diluting the concentrated mixture to produce a diluted mixture having a second cell titer; and
(d) subjecting the diluted mixture to conditions suitable for growth of the phototrophic organism in the absence of the wavelength converting material.

2. A method comprising:
(a) transforming a plurality of at least one phototrophic organism with a construct comprising a promoter having a sequence selected from the group consisting of SEQ ID No. 1, and SEQ ID No. 2, to generate a transformed phototrophic organism;
(b) introducing the transformed phototrophic organism to a media to create a first mixture;
(c) subjecting the first mixture to conditions suitable for growth of the transformed phototrophic organism in the presence of a wavelength converting material to produce a concentrated mixture having a first cell titer;
(d) diluting the concentrated mixture to produce a diluted mixture having a second cell titer; and
(e) subjecting the diluted mixture to conditions suitable for growth of the transformed phototrophic organism in the absence of the wavelength converting material.

3. The method of claim 2 wherein the promoter is operably linked to a second polynucleotide.

4. The method of claim 2 wherein the promoter is induced in the absence of the wavelength conversion material.

5. The method of claim 4 wherein induction of the promoter results in an increased expression of the second polynucleotide.

6. The method of claim 5 wherein expression of the second polynucleotide is increased by from about 2-fold to about 7-fold.

7. The method of claim 5 wherein the increased expression of the second polynucleotide results in the expression of a target product.

8. The method of claim 7 wherein the target product comprises a hydrocarbon.

9. The method of claim 1 wherein the phototrophic organisms comprise algae, euglena,
phytoplankton, bacteria, or combinations thereof.

10. The method of claim 1 wherein the phototrophic organism is selected from the group consisting of Synechocystis sp. PCC 6803, Anabaena sp. PCC 7120, Thermosynechococcus elongatus BP-1, Gloeobacter violaceus PCC 7421, Microcystis aeruginosa NIES-843, Prochlorococcus marinus SS120, Prochlorococcus marinus MED4, Prochlorococcus marinus MIT9313, Synechococcus sp. WH8102, Synechococcus elongatus PCC 6301, Synechococcus sp. CC9311, Synechococcus sp. PCC 7002, Acaryochloris marina MBIC11017, Prochlorococcus marinus str. NATL2A, Anabaena variabilis ATCC 29413, Synechococcus sp. CC9902, Synechococcus sp. CC9605, Prochlorococcus marinus str. MIT 9312, Synechococcus elongatus PCC 7942, Synechococcus sp. JA-2-3B′a(2-13), Synechococcus sp. JA-3-3Ab, Prochlorococcus marinus str. AS9601, Prochlorococcus marinus str. MIT 9515, Prochlorococcus marinus str. MIT 9303, Prochlorococcus marinus str. NATL1A, Prochlorococcus marinus str. MIT 9301, Synechococcus sp. RCC307, Synechococcus sp. WH 7803 Prochlorococcus marinus str. MIT 9215, Prochlorococcus marinus str. MIT 9211, Cyanothece sp. ATCC 51142, Nostoc punctiforme ATCC 29133, Chlorobium tepidum TLS, Rhodopseudomonas palustris CGA009, Trichodesmium erythraeum IMS101, Cyanothece sp. PCC 7424, Cyanothece sp. PCC 7425, Cyanothece sp. PCC 8801, Arthrospira platensis NIES-39, and combinations thereof.

11. The method of claim 1 wherein the wavelength conversion material comprises an organic fluorescent dye and a polymeric matrix, wherein the organic fluorescent dye is solubilized in the polymeric matrix, and wherein the wavelength-conversion material is capable of absorbing light comprising a wavelength of 280 to 650 nm and emitting the absorbed light at a wavelength of 400 to 800 nm.

12. The method of claim 11 wherein the organic fluorescent dye is a perylene-containing compound.

13. The method of claim 11 wherein the organic fluorescent dye is a coumarin dye, a carbocyanine dye, a phthalocyanine dye, an oxazine dye, a carbostyryl dye, a porphyrin dye, an acridine dye, an anthraquinone dye, an arylmethane dye, a quinone imine dye, a thiazole dye, a bis-benzoxazolylthiophene (BBOT)dye, or a xanthene dye, or any combination of dyes thereof.

14. The method of claim 11 wherein the polymeric matrix comprises a polycarbonate, a polyolefin such as polyethylene, a polymethyl (meth)acrylate, a polyester, an elastomer, a polyvinyl alcohol, a polyvinyl butyral, polystyrene, or a polyvinyl acetate, or any combination or copolymer thereof.

15. A method comprising: exposing, for a first period of time, a phototrophic organism to modified natural or artificial sunlight, wherein the modified sunlight is shifted toward the red spectrum by passing the sunlight through a medium comprising a luminescent dye; and exposing, for a second period of time, the phototrophic organism to non-modified natural or artificial sunlight whereby the phototrophic organism is induced to express a desired product.

16. The method of claim 15 wherein the exposing for the first period of time is in a first bioreactor, wherein at least a portion of the first bioreactor is formed from a polymer comprising the dye; and wherein the exposing for the second period of time is in a second bioreactor, wherein the first bioreactor is formed from a polymer substantially free of the dye.

17. The method of claim 15 wherein the first period of time is an incubation period associated with transfer of the phototrophic organism to a location receiving direct sunlight.

18. The method of claim 17 wherein the incubation period is further associated with a diluted culture of the phototrophic organism such that the phototrophic organism is incapable of self-shading and susceptible to culture crash.

19. The method of claim 18 wherein the start of the second period of time is associated with a achieving a non-diluted culture of the phototrophic organism such that the phototrophic organism is capable of self-shading and less susceptible to culture crash than during the first period of time.

20. The method of claim 15 wherein the phototrophic organism photosynthetic efficiency is at least 1×, 2×, or 3× greater in the first 50 hours after inoculation than in an otherwise similar first bioreactor absent the dye.


SEQUENCE LISTING (46 sequences)

SEQ ID NO: 1 - DNA, 218 bp, Artificial Sequence
  Sequence of light inducible promoter for the A2740 gene of Synechococcus PCC7002
ttggtacgggcctgtgtcggtttgggatgtttcaagcggtgcggggcgagggcaacccagtaactaaaaatcggacaacccagcaggctatttcccgctagatgaatccctccaaaaagttttgggaaaagggttgacatctcaaaattcttcagttagattgtataactgtgagtcgcaagcgaaacaaacaaaagcgcttctgctcagtccgcaag

SEQ ID NO: 2 - DNA, 224 bp, Artificial Sequence
  Sequence of light inducible promoter for the A0312 gene of Synechococcus PCC7002
taaatctaaatgtcgtttcccctgggaaatatcttttcaaaaaatagcaaatttagcctttaatcaagttcaaccttggccaggacaattgcaactttatccttagagaggcaaggttttcttaaggttttctaagagaatcttaaatatatcaggataaatattcacagccttggggttggtactagcatgggacaaagacccgactttaggctgacaaaata

SEQ ID NO: 3 - DNA, 224 bp, Artificial Sequence; identical to SEQ ID NO: 2
  Sequence and primers for Synechococcus PCC7002 A2137 gene upstream sequence

SEQ ID NO: 4 - DNA, 43 bp, Artificial Sequence
  Primer for Synechococcus PCC7002 A2137 gene upstream sequence
gcattgttgatcctgaaacgcagttgctcggcgatcgcctgga

SEQ ID NO: 5 - DNA, 44 bp, Artificial Sequence
  Sequence and primers for Synechococcus PCC7002 A2137 gene upstream sequence
aaaaccatatgtttctcctcttctggcgtgacgataaatgattg

SEQ ID NO: 6 - DNA, 477 bp, Artificial Sequence
  Sequence and primers for Synechococcus PCC7002 D0016 gene upstream sequence
gcattgttgatcctgaaacgcttccaagtgtccttgtcgtactcagcgtgggtcaaaaaatatttgtagtaaacggtttgagatgggtagtcgatgccaacgattaagcgatatttattccccttgatgttaaatacagtaaaatttccgacggcctccgcgtctcgataggttttccgaacctcttctagattttgccattgggcctgcttgatcactgcataccaagcatctagggctgatttactatcagagtatttggctgcatcttgccttagtttgctgatgctgattaagtgcatatagtccttttccccaatagtaaccgaatactctcaaaatgagagtatttaataacaacttccagggctgttgtccaggttaataaagtggtaactatcgccatacattgccacgtggacaaatgtttcagtcccgtagttgggatttagtggtaagaggagaaacatatggttt

SEQ ID NO: 7 - DNA, 43 bp, Artificial Sequence
  primers for Synechococcus PCC7002 D0016 gene upstream sequence
gcattgttgatcctgaaacgcttccaagtgtccttgtcgtact

SEQ ID NO: 8 - DNA, 41 bp, Artificial Sequence
  primers for Synechococcus PCC7002 D0016 gene upstream sequence
aaaccatatgtttctcctcttaccactaaatcccaactacg

SEQ ID NO: 9 - DNA, 451 bp, Artificial Sequence
  Sequence and primers for Synechococcus PCC7002 D0011 gene upstream sequence
gcattgttgatcctgaaacgctttaacatggtctacaactgaactatttcaagttgtttcatccacaaaatctgttttatggcatggcttctaatgaggcttatatctaagacatctactgtcttagacttctctccatttcagtggtcatgctgaccatacaaatatttcagtccccttttagagatttactccaaatttctttcttctttgagcaatcacaattgtcatcaagagtattcccaacatcagggacaaaagttctctaataatcattgatgagggcgatcgccaacacattcaaaaatttctgccattttctgacagaagaagtccccagccgattgtggatattttgcgcttcgtcaaaaaaaccttccagaggtgtataaggtgtggggaaatttcctcggcttgaccagaaggtaataagaggagaaacatatggttt

SEQ ID NO: 10 - DNA, 45 bp, Artificial Sequence
  Primers for Synechococcus PCC7002 D0011 gene upstream sequence
gcattgttgatcctgaaacgctttaacatggtctacaactgaact

SEQ ID NO: 11 - DNA, 45 bp, Artificial Sequence
  Primers for Synechococcus PCC7002 D0011 gene upstream sequence
aaaccatatgtttctcctcttattaccttctggtcaagccgagga

SEQ ID NO: 12 - DNA, 579 bp, Artificial Sequence
  Sequence and primers for Synechococcus PCC7002 D0017 gene upstream sequence
gcattgttgatcctgaaacgctatttggctgcatcttgccttagtttgctgatgctgattaagtgcatatagtccttttccccaatagtaaccgaatactctcaaaatgagagtatttaataacaacttccagggctgttgtccaggttaataaagtggtaactatcgccatacattgccacgtggacaaatgtttcagtcccgtagttgggatttagtggttggaaagcgccactgaagcaaacgtgagcaacgcggaagccaagtttcagtcccgtaatcgggatttagtggttggaaagcagcccacccaagatatccccgtccgtgggtcttgtttcagtcccgtaatcgggatttagtggttggaaagtctgccacaaaaatcaaagcccccattttttcttgtttcagtcccgtaatcgggatttagtggttggaaagtcggcgaggtcaccaaggtcggctcctgccgccgtgattgaagtttcagtcccgtaatcgggatttagtggttggaaaggcgggaacaactttgatgcgatccgaagtaccaataagaggagaaacatatggttt

SEQ ID NO: 13 - DNA, 44 bp, Artificial Sequence
  Primers for Synechococcus PCC7002 D0017 gene upstream sequence
gcattgttgatcctgaaacgctatttggctgcatcttgccttag

SEQ ID NO: 14 - DNA, 42 bp, Artificial Sequence
  Primers for Synechococcus PCC7002 D0017 gene upstream sequence
aaaccatatgtttctcctcttattggtacttcggatcgcatc

SEQ ID NO: 15 - DNA, 300 bp, Artificial Sequence
  Sequence and primers for Synechococcus PCC7002 petM gene upstream sequence
gcattgttgatcctgaaacgctagttcctgtagctaaaatcgctcatcatagcaaaaaacaaaaactatataatcccacaaaaaccccacaaatttggagtttggagtttatttttttaatttggagaaatttgcagagcggcgatcgcccctggaatatcccaaccgcgcccctggaataccccaaccgcaaaaatcccactacctgctttaaaatgtcctttgtggccaaaccgctttaaaatcaagccattataaccagacaaaggagtatttccaaagaggagaaacatatggttt

SEQ ID NO: 16 - DNA, 45 bp, Artificial Sequence
  Primers for Synechococcus PCC7002 petM gene upstream sequence
gcattgttgatcctgaaacgctagttcctgtagctaaaatcgctc

SEQ ID NO: 17 - DNA, 48 bp, Artificial Sequence
  Primers for Synechococcus PCC7002 petM gene upstream sequence
aaaccatatgtttctcctctttggaaatactcctttgtctggttataa

SEQ ID NO: 18 - DNA, 259 bp, Artificial Sequence
  Sequence and primers for Synechococcus PCC7002 A2740 gene upstream sequence
gcattgttgatcctgaaacgcttggtacgggcctgtgtcggtttgggatgtttcaagcggtgcggggcgagggcaacccataactaaaaatcggacaacccagcaggctatttcccgctagatgaatccctccaaaaagttttgggaaaagggttgacatctcaaaattcttcagttagattgtataactgtgagtcgcaagcgaaacaaacaaaagcgcttctgctcagtccgcaagaagaggagaaacatatggttt

SEQ ID NO: 19 - DNA, 46 bp, Artificial Sequence
  Primers for Synechococcus PCC7002 A2740 gene upstream sequence
gcattgttgatcctgaaacgcttggtacgggcctgtgtcggtttgg

SEQ ID NO: 20 - DNA, 42 bp, Artificial Sequence
  Primers for Synechococcus PCC7002 A2740 gene upstream sequence
aaaccatatgtttctcctcttcttgcggactgagcagaagcg

SEQ ID NO: 21 - DNA, 300 bp, Artificial Sequence
  Sequence and primers for Synechococcus PCC7002 isp gene upstream sequence
gcattgttgatcctgaaacgcttttgtggtctgatcgctctccacagttgcggcattctttatctgatggggctgaccctcttagaaaaattacaggcgacggaactgagtttgctccaggcgatcgccctctatagtgtgactccctttccgagtcagttaatgctagtctgtggtgtggccgtggtggcctatggggtacgacggattctcttctattaagccaaagcattgggcttgatattttttctttttagcgaaacggaaactctctctccaaagaggagaaacatatggttt

SEQ ID NO: 22 - DNA, 45 bp, Artificial Sequence
  Primers for Synechococcus PCC7002 isp gene upstream sequence
gcattgttgatcctgaaacgcttttgtggtctgatcgctctccac

SEQ ID NO: 23 - DNA, 48 bp, Artificial Sequence
  Primers for Synechococcus PCC7002 isp gene upstream sequence
aaaccatatgtttctcctctttggagagagagtttccgtttcgctaaa

SEQ ID NO: 24 - DNA, 457 bp, Artificial Sequence
  Sequence and primers for Synechococcus PCC7002 tig gene upstream sequence
gcattgttgatcctgaaacgcaagaataaacctcctaatgactttgggttaagggtaatgaaattaaaaagtaaaaatctggttggggcgatcgcctagggggaatttttcactgtggcggggctggcttgatcaccccagaggcgagggtttggctaggataagcagggccacttttgtcgttctgtgctggggcatccctttcgcaagactttgtatatcgaagctctaattatactattatcagataggttaacccctcgtaaaaacgcaatagaccgacccaaacatattctgggtaatcctgcgtttgggttactgaccttaggatatcatggagcgctcaattcagacccctaaaggggcgatcgccaacgaacagaactatcctaagacccttagttgtaacttatatattagaacctttaaacatcgaaagaggagaaacatatggttt

SEQ ID NO: 25 - DNA, 46 bp, Artificial Sequence
  Primers for Synechococcus PCC7002 tig gene upstream sequence
gcattgttgatcctgaaacgcaagaataaacctcctaatgactttg

SEQ ID NO: 26 - DNA, 44 bp, Artificial Sequence
  Primers for Synechococcus PCC7002 tig gene upstream sequence
aaaccatatgtttctcctctttcgatgtttaaaggttctaatat

SEQ ID NO: 27 - DNA, 266 bp, Artificial Sequence
  Sequence and primers for Synechococcus PCC7002 A3012 gene upstream sequence
gcattgttgatcctgaaacgctaaatctaaatgtcgtttcccctgggaaatatcttttcaaaaaatagcaaatttagcctttaatcaagttcaaccttggccaggacaattgcaactttatccttagagaggcaaggttttcttaaggttttctaagagaatcttaaatatatcaggataaatattcacagccttggggttggtactagcatgggacaaagacccgactttaggctgacaaaataaagaggagaaacatatggttt

SEQ ID NO: 28 - DNA, 48 bp, Artificial Sequence
  Primers for Synechococcus PCC7002 A3012 gene upstream sequence
gcattgttgatcctgaaacgctaaatctaaatgtcgtttcccctggga

SEQ ID NO: 29 - DNA, 50 bp, Artificial Sequence
  Primers for Synechococcus PCC7002 A3012 gene upstream sequence
aaaccatatgtttctcctctttattttgtcagcctaaagtcgggtctttg

SEQ ID NO: 30 - DNA, 343 bp, Artificial Sequence
  Sequence and primers for Synechococcus PCC7002 psaE gene upstream sequence
gcattgttgatcctgaaacgcgatcccctcacccaactctttggtatggggactgctgctgccacagcctgggcgatcgcccctgatctcggtaaatatgcccaacagcacatcaccaccgcgattcccctcaatctcgccctcgaagaaatccaagaaaacacggacgagagtggaaatccaagccattttgtccgattacggtggcaggacgcgcaataaaacttaaacacttcaagtgcctgaaaacccctatccgatagtaggctgttatctaagtctaactacactctttagtaatcgctttaaaggaacataagcaaagaggagaaacatatggttt

SEQ ID NO: 31 - DNA, 343 bp, Artificial Sequence; identical to SEQ ID NO: 30
  Primers for Synechococcus PCC7002 psaE gene upstream sequence

SEQ ID NO: 32 - DNA, 47 bp, Artificial Sequence
  Primers for Synechococcus PCC7002 A3012 gene upstream sequence
aaaccatatgtttctcctctttgcttatgttcctttaaagcgattac

SEQ ID NO: 33 - DNA, 741 bp, Artificial Sequence
  synthetic sequence
aagaggagaaacatatggtttctaaaggcgaagaactgttcactggtgtagtacccatcctcgtggaactggatggtgacgtaaacgggcacaaattttccgtatccggtgagggtgaaggtgatgccacctatggcaaattgaccctgaaatttatctgcaccacgggtaaacttccggtcccctggcctaccctggttaccaccctaacttatggcgtacagtgctttgctagataccccgaccacatgaaacaacatgactttttcaaatccgccatgcccgaaggctacgtacaggaacggaccatttttttcaaagatgatggtaattacaaaacacgcgccgaagtcaaatttgaaggcgacaccttggtcaaccgcatcgaattgaaaggcattgacttcaaagaggatggcaacattttgggccacaagttagaatataattataacagccacaacgtctacattaccgccgataaacaaaaaaatggcattaaagcgaattttaagattcggcacaacattgaggacgggggtgtacaactcgctgatcactatcagcagaataccccaatcggggacggtcccgtcctcttgcccgacaatcactacttgtccacccagagtaagttgagcaaggaccccaatgaaaaacgggatcacatggtattgctcgagtttgtcaccgccgctggcattactctcggtatggacgagttatataaataatggatcc

SEQ ID NO: 34 - DNA, 33 bp, Artificial Sequence
  synthetic sequence
aagaggagaaacatatggtttctaaaggcgaag

SEQ ID NO: 35 - DNA, 43 bp, Artificial Sequence
  synthetic sequence
gacacaacgtggctttggatccattatttatataactcgtcca

SEQ ID NO: 36 - DNA, 48 bp, Artificial Sequence
  Primers for the Kanamycin Resistance gene(KmR)
agttatataaataatggatccaaagccacgttgtgtctcaaaatctct

SEQ ID NO: 37 - DNA, 43 bp, Artificial Sequence
  Primers for the Kanamycin Resistance gene(KmR)
ccgccattcggattgattagaaaaactcatcgagcatcaaatg

SEQ ID NO: 38 - DNA, 929 bp, Artificial Sequence
  Sequence and primers for the Kanamycin Resistance gene(KmR)
aaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatca

SEQ ID NO: 39 - DNA, 743 bp, Artificial Sequence
  Sequence and primers for the Kanamycin Resistance gene(KmR)
atccgaatggcggaatcttcgggtattgatgctaccctgaaaatattttgtagatagggcccctttagcccactgatttaacaaaaccgagttacatttctctaggaaaacattcacatggacggaaaccaagtgcctactgttaccctcaaacgcccaattaatctgaaggtcatcgtgaccccccgctggaaagaggaagtacagcagcaactccagggacaaatagcgacttttgatggtcaattacaacaactggatgtccaaggaaatcgcaccattagtgaacttcaacaacagggcaataccccccaggtcgctcaacaaatggaaaatatccaactccaggttaaccaaaaaaagcgggaactccttgagaagaaaaaccaagtgctccagcagctccaacaggtacaaaacttagaattagatcaagagtttctccagggtcaaatggagagtttctttgaagtgaaagtgggcgataatctggtgcgcaagctgaatgttgaacttgtggtccgggatggtgttgttgaagaaattcgcggcgacctctaattttttttgtgattgctgtcgagtgacagtgttcggctcccccgatgacacttgcggtgcgggggatttttaatggggaaaaaattcatgaaaattggcattgtcgggctgggtttaatcggtggatctttggcgatcgcctttcgggaaaaaggcttagaagtgttgggagtttctcggaa

SEQ ID NO: 40 - DNA, 26 bp, Artificial Sequence
  Primers for flanking sequences for inserting genes at the Synechococcus 7002 fadD genome locus.
gccccaagatttgatgaccttgatct

SEQ ID NO: 41 - DNA, 28 bp, Artificial Sequence
  synthetic sequence
gcgtttcaggatcaacaatgcgaatttc

SEQ ID NO: 42 - DNA, 25 bp, Artificial Sequence
  Primers for flanking sequences for inserting genes at the Synechococcus 7002 fadD genome locus.
aacgcaatgtgtgtggttctgctgg

SEQ ID NO: 43 - DNA, 43 bp, Artificial Sequence
  Primers for flanking sequences for inserting genes at the Synechococcus 7002 fadD genome locus.
cgatgagtttttctaatcaatccgaatggcggaatcttcgggt

SEQ ID NO: 44 - DNA, 28 bp, Artificial Sequence
  Primers for flanking sequences for inserting genes at the Synechococcus 7002 fadD genome locus.
ttccgagaaactcccaacacttctaagc

SEQ ID NO: 45 - DNA, 26 bp, Artificial Sequence
  Primers for flanking sequences for inserting genes at the Synechococcus 7002 fadD genome locus.
gtgggctaaaggggccctatctacaa

SEQ ID NO: 46 - DNA, 741 bp, Artificial Sequence; identical to SEQ ID NO: 33
  Sequence for SFGP gene